SEQUENCE LISTING (2 sequences)

SEQ ID NO: 1 - protein, 436 aa, Artificial Sequence
  Synthetic Construct
MAKYSELEKGGVIMQAFYWDVPSGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 2 - protein, 436 aa, Artificial Sequence
  Synthetic Construct
MAKYSELEEGGLIMQAFYWDVPMGGIWWDTIAQKIPDWASAGISAIWIPPASKGMGGAYSMGYDPYDFFDLGEYDQKGTVETRFGSKQELVNMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNNNIPALVDALRYGQTVVSRDPFKAVTFVANHDTDIIWNKYPAYAFILTYEGQPTIFYRDYEEWLNKDKLKNLIWIHDNLAGGSTDIVYYDNDELIFARNGYGSKPGLITYINLASSKAGRWVYVPKFAGSCIHEYTGNLGGWVDKWVDSSGWVYLEAPAHDPANGQYGYSVWSYCGVG

(19) United States
(12) Patent Application Publication
(10) Pub. No.: US 2023/0323327 A1
(43) Pub. Date: Oct. 12, 2023

(54) ALPHA-AMYLASE VARIANTS

(71) Applicant: BASF SE, Ludwigshafen am Rhein (DE)

(72) Inventors: Asfia Qureshi, San Diego, CA (US); Priya Anand, Ludwigshafen am Rhein (DE); Jared Dennis, San Diego, CA (US); Hugo Urbina, San Diego, CA (US); Gaetano Speciale, San Diego, CA (US)

(21) Appl. No.: 18/026,807

(22) PCT Filed: Sep. 2, 2021
(86) PCT No.: PCT/US2021/048799

§ 371 Date: Mar. 16, 2023

Related U.S. Application Data
(60) Provisional application No. 63/081,695, filed on Sep. 22, 2020.

(51) Int. Cl. C12N 9/28

(52) U.S. Cl. CPC C12N 9/2417

(57) ABSTRACT

The present invention relates to variants of an alpha-amylase which have an increased solubility at pH 6.0, an increased solubility at pH 10.0, a higher resistance to protein aging, and/or an increased specific activity compared to the parent alpha-amylase. The present invention also relates to methods of making the variant alpha-amylase and the use of the variant alpha-amylase in processing starch, cleaning or washing textiles, hard surfaces, or dishes, making ethanol, treating an oil well, processing pulp or paper, animal feed, syrup production, and preparing a dough or a baked product prepared from the dough.

### FIELD OF THE INVENTION

The present invention relates to variants of an alpha-amylase which have an increased solubility at pH≤6.0, an increased solubility at pH≥10.0, a higher resistance to protein aging, and/or an increased specific activity compared to the parent alpha-amylase. The present invention also relates to methods of making the variant alpha-amylase and the use of the variant alpha-amylase in starch processing, cleaning or washing textiles, hard surfaces, or dishes, making ethanol, treating an oil well, processing pulp or paper, animal feed, syrup production, preparing a dough or a baked product prepared from the dough, and in a detergent or personal care product.

### BACKGROUND OF THE INVENTION

Alpha-amylases are used in animal feed, detergents, personal care products, processing of textiles, pulp and paper processing, in ethanol production, in lignocellulosic ethanol production, in syrups production, in the baking industry, or as viscosity breakers in oilfield and mining industries.

However, many available alpha-amylases have limited pH-ranges at which they are active and soluble, limiting the reactions and environments they can be utilized in, thereby making high concentration formulation difficult. They further are susceptible to protein aging, such as oxidation over time, leading to decreased activity and limiting their shelf-life. Similarly, limited specific activity ranges lead to the need to use relatively large amounts of enzyme, which is expensive. For example, commercially available alpha-amylases can suffer from low solubility at low and high pH (e.g. pH 6.0 and 10.0). Commercial alpha-amylases can also be oxidized over time, limiting their shelf-life.

Accordingly, there is a need for alpha-amylases that exhibit high solubility at or below pH 6.0, high solubility at or above pH 10.0, a high resistance to protein aging, and/or high specific activity.

One solution to this problem are the inventive variant polypeptides having alpha-amylase enzyme activity that meet or exceed these industrial requirements and exhibit an increased solubility at or below pH 6.0, an increased solubility at or above pH 10.0, a higher resistance to protein aging, and/or an increased specific activity compared to the alpha-amylase according to SEQ ID No. 1 or SEQ ID No. 2.

### SUMMARY OF THE INVENTION

The present inventors found that introducing amino acid modifications in the amino acid sequence of an alpha-amylase increases the solubility at pH≤6.0 and/or pH≥10.0, increases the resistance protein aging, and/or increases the specific activity compared to the parent alpha-amylase.

Accordingly, the present invention relates to a variant polypeptide of the alpha-amylase according to SEQ ID No. 1 or SEQ ID No. 2 having alpha-amylase activity and comprising an amino acid sequence which is at least 80% identical to the sequence according to SEQ ID No. 1, which amino acid sequence comprises
a) at least one amino acid modification at an amino acid residue position number selected from the group consisting of: 23, 33, 125, 133, 157, 181, 214, 228, 260, 272, 323, 336, 349, 357, 407, and 408 or a combination thereof in the numbering of SEQ ID No. 1, or
b) at least one amino acid modification at an amino acid residue position number selected from the group consisting of: 55 and 61 or a combination thereof in the numbering of SEQ ID No. 1, or
c) amino acid modifications at amino acid residue position numbers 205 and 206 in the numbering of SEQ ID No. 1, or a combination thereof.

In one embodiment, the amino acid modification(s) is/are an amino acid substitution, insertion, deletion, or any combination thereof.

In one embodiment, the amino acid modification(s) is/are an amino acid substitution, and the amino acid substitution is a conservative amino acid substitution.

In one embodiment,
(a) the at least one amino acid modification is an amino acid substitution selected from the group consisting of: S23E or M23E, Q33E, N125E or G125E, S133T, S157D, Q181E, N214D, N228S, N260D/E, Q272D, N323E, N336A, N349P, N357E, S407E and S408E or a combination thereof in the numbering of SEQ ID No. 1, preferably S407E, or
(b) the at least one amino acid modification is an amino acid substitution selected from the group consisting of: M55A/L/Q and M61V or a combination thereof in the numbering of SEQ ID No. 1, preferably M55L, or
(c) the amino acid modifications are A205G and P206A in the numbering of SEQ ID No. 1.

In one such embodiment, the variant polypeptide comprises the amino acid modifications of:
a) S23E and N125E, or
b) Q33E and S157D, or
c) Q181E and N260D, or
d) N214D and S407E, or
e) S23E, Q181E, N260D, Q272E, N323E, N349P, N357E, S407E, and S408D, or
f) S23E, Q181E, N228S, N260D, Q272E, N323E, N349P, N357E, and S408D, or
g) S23E, Q33E, Q181E, N260E, Q272D, N323E, N349P, N357E, and S407E, or
h) S23E, N260E, Q272E, and S407E, or
i) S23E, N214D, N260E, Q272E, and S407E, or
J) S23E, N214D, N260D, Q272E, and S407E, or
k) S23E, N260E, Q272E, N349P, and S407E, or
l) S23E, N260E, Q272E, and S407E, or
m) M55A and M61V, or
n) M55L and M61V, or
o) M55Q and M61V, or
p) A205G, P206A, and S407E, or
q) K9E, V12L, S23M, R31A, E37D, Y39A, D40S, M1021, N125G, K259N, S266D, Q269R, N270Y, E335D, V355A, D361S, G372A, A388A, Y404W, Y406D, S407E, and Y418H, or
r) K9E, V12L, S23M, R31A, E37D, Y39A, D40S, M1021, N125G, A205G, P205A, K259N, S266D, Q269R, N270Y, E335D, V355A, D361S, G372A, A388A, Y404W, Y406D, and Y418H in the numbering of SEQ ID No. 1

In one embodiment, the variant polypeptide comprises at least one amino acid modification at an amino acid residue position number selected from the group consisting of: 23, 33, 125, 133, 157, 181, 214, 228, 260, 272, 323, 334, 349, 357, 407, and 408 or a combination thereof in the numbering of SEQ ID No. 1 and has an increased solubility at pH≤6.0 compared to the polypeptide of SEQ ID No. 1 or SEQ ID No. 2.

In one embodiment, the variant polypeptide comprises at least one amino acid modification at an amino acid residue position number selected from the group consisting of: 23, 33, 125, 133, 157, 181, 214, 228, 260, 272, 323, 336, 349, 357, 407, and 408 or a combination thereof in the numbering of SEQ ID No. 1 and has an increased solubility at pH 10.0 compared to the polypeptide of SEQ ID No. 1 or SEQ ID No. 2.

In one embodiment, the variant polypeptide comprises at least one amino acid modification at an amino acid residue position number selected from the group consisting of: 55 and 61 or a combination thereof in the numbering of SEQ ID No. 1 and is more resistant to protein aging compared to the polypeptide of SEQ ID No. 1 or SEQ ID No. 2.

In one embodiment, the variant polypeptide comprises amino acid modifications at amino acid residue position numbers 205 and 206 in the numbering of SEQ ID No. 1 and has increased specific activity compared to the polypeptide of SEQ ID No. 1 or SEQ ID No.2.

In one embodiment, the variant polypeptide has alpha-amylase activity and is a fragment of the full-length amino acid sequence.

In one embodiment, the variant polypeptide comprises a hybrid of at least one variant polypeptide according to any one of the preceding embodiments and a second polypeptide having amylase activity, wherein the hybrid has alpha-amylase activity.

The present invention further relates to a composition comprising the variant polypeptide according to any one of the preceding embodiments.

In one embodiment, the composition further comprises a second enzyme.

In one such embodiment, the second enzyme is selected from the group consisting of: a beta-amylase, a lipase, a second alpha-amylase, a G4-amylase, a xylanase, a protease, a cellulase, a glucoamylase, an oxidoreductase, a phospholipase, and a cyclodextrin glucanotransferase.

The present invention further relates to a method of making a variant polypeptide comprising: providing a template nucleic acid sequence encoding the inventive variant polypeptide, transforming the template nucleic acid sequence into an expression host, cultivating the expression host to produce the variant polypeptide, and purifying the variant polypeptide.

In one such embodiment, the expression host is selected from the group consisting of: a bacterial expression system, a yeast expression system, a fungal expression system, and a synthetic expression system.

In one such embodiment, the bacterial expression system is selected from an E. coli, a Bacillus, a Pseudomonas, and a Streptomyces.

In another such embodiment, the yeast expression system is selected from a Candida, a komagataella, a Saccharomyces, a Schizosaccharomyces.

In another such embodiment, the fungal expression system is selected from a Penicillium, an Aspergillus, a Fusarium, a Myceliopthora, a Rhizomucor, a Rhizopus, a Thermomyces, and a Trichoderma.

The present invention further relates to a use of the inventive variant polypeptide for starch processing.

The present invention further relates to a use of the inventive variant polypeptide for cleaning or washing textiles, hard surfaces, or dishes.

The present invention further relates to a use of the inventive variant polypeptide for making ethanol.

The present invention further relates to a use of the inventive variant polypeptide for treating an oil well.

The present invention further relates to a use of the inventive variant polypeptide for processing pulp or paper.

The present invention further relates to a use of the inventive variant polypeptide for animal feed.

The present invention further relates to a use of the inventive variant polypeptide for syrup production.

The present invention further relates to a use of the inventive variant polypeptide for preparing a dough or a baked product prepared from the dough.

The present invention further relates to a use of the inventive variant polypeptide in a detergent or personal care product.

### BRIEF DESCRIPTION OF THE DRAWINGS

FIG. 1: Solubility determination of variants after NH4SO4 precipitation.

FIG. 2: Solubility determination after Heat Treatment.

FIG. 3: Percentage loss of specific activity of alpha-amylase variants upon oxidation.

FIG. 4: Specific activity of alpha-amylase variant 40 compared to the alpha-amylase according to SEQ ID No. 2.

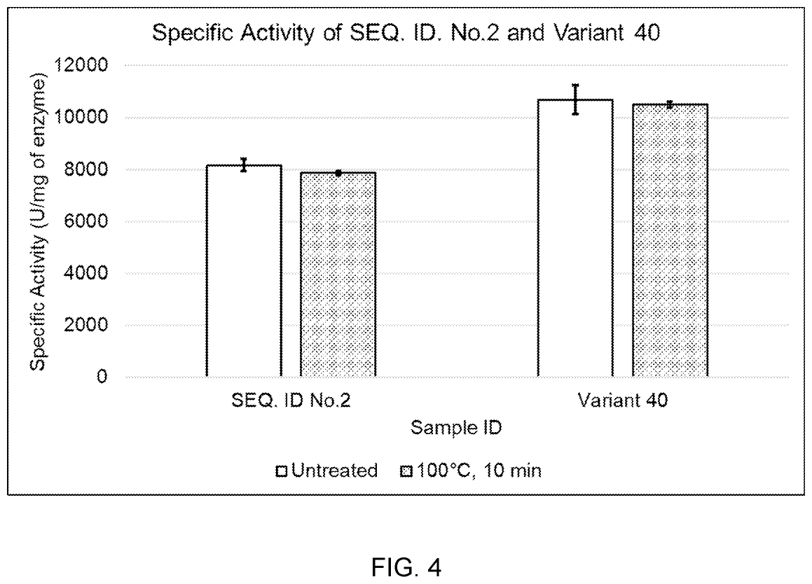

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As discussed above, the present invention is based on the finding that variants of an alpha-amylase have an increased solubility at pH≤6.0, an increased solubility at pH≥10.0, a higher resistance to protein aging, and/or an increased specific activity compared to the parent alpha-amylase.

A “variant polypeptide” refers to an enzyme that differs from its parent polypeptide in its amino acid sequence. A “variant alpha-amylase” refers to an alpha-amylase that differs from its parent alpha-amylase in its amino acid sequence and has alpha-amylase activity. Variant polypeptides are described using the nomenclature and abbreviations for single amino acid molecules according to the recommendations of IUPAC for single letter or three letter amino acid abbreviations.

A “parent” polypeptide amino acid sequence is the starting sequence for introduction of amino acid modifications (e.g. by introducing one or more amino acid substitutions, insertions, deletions, or a combination thereof) to the sequence, resulting in “variants” of the parent polypeptide amino acid sequence. A parent polypeptide includes both a wild-type polypeptide amino acid sequence or a synthetically generated polypeptide amino acid sequence that is used as starting sequence for the introduction of (further) changes. Within the present invention the parent polypeptide is preferably the polypeptide having the amino acid sequence according to SEQ ID No. 1. Alternatively, the parent polypeptide may be a polypeptide comprising an amino acid sequence which is at least 90% identical to the amino acid sequence according to SEQ ID No. 1 and which does not have an amino acid modification at any of the following amino acid residues: 13, 25, 27, 90, 91, 131, 132, 148, 185, 196, 198, 205, 206, 208, 209, 210, 214, 220, 222, 236, 239, 251, 269, 276, 318, 364, 369, 375, 389, 419, 435, 438, 463, 469, 494, 499, 502, and 519 compared to the sequence according to SEQ ID No. 1. Alternatively, the parent polypeptide has the amino acid sequence of SEQ ID No. 2, which is 95% identical to the amino acid sequence of SEQ ID No. 1 and comprises amino acid modification at positions 9, 12, 23, 32, 37, 39, 40, 102, 125, 259, 266, 269, 270, 335, 355, 361, 372, 388, 404, 406, and 418 compared to the sequence according to SEQ ID No. 1.

Alpha-amylases (E.C. 3.2.1.1), also known as 1,4-α-D-glucan glucanohydrolases or glycogenases, are enzymes that perform hydrolysis of random (1->4)-alpha-D-glucosidic linkages in polysaccharides such as starch and glycogen. Alpha amylases are widely used in industrial settings, e.g. to break starches in grains down into fermentable sugars, to treat cornstarch in the production of high-fructose corn syrup, in detergents such as dishwashing and starch-removing detergents.

Alpha-amylases are characterized in animals, plants and microbial sources. Commercial alpha-amylase enzymes used in foods, feeds, desizing of textiles, the paper industry, starch saccharification, detergents, and baking include Amzyme TX from Parchem, Aquazym 1201, Aquazym Ultra 2501, and Thermamyl®, Takaterm from Novo Nordisk, BAN™, Liquozyme® SCDC, Natalase®, and Stainzyme® plus from Novozymes, Enzymex (Cocktail) from Exotic Biosolutions Pvt. Ltd., Fructamyle FHT from ERBSLOEH, Validase BAA from DSM Valley Research, FUELZYME® from BASF, and Veron® from AB Enzymes.

The alpha-amylase activity can be determined by various assays known to the person skilled in the art, including reducing end assays, starch specific assays, and colorimetric assays using artificial substrates. Examples of those are the PAHBAH assay (Lever (1972) Anal. Biochem. 47: 273-279) the DNS assay (Miller (1959) Anal. Chem. 3:426-428), the MBTH assay (Barrett (2002) Anal. Biochem. 305:287-289), the starch-iodine assay (Fuwa (1954) J. Biochem. 41: 583-603), the Betamyl-3 and the red starch assays available from Megazyme, the Phadebas® Amylase test, and the Infinity Amylase available from ThermoFisher.

The variant polypeptides of the present invention are characterized in that they have an increased solubility at pH≤6.0, an increased solubility at pH≥10.0, a higher resistance to protein aging, and/or an increased specific activity compared to the parent alpha-amylase.

“Solubility” is the ability of a solid, liquid, or gaseous chemical substance (referred to as the solute) to dissolve in solvent (usually a liquid) and form a solution. An increased solubility at a certain pH means that the variant polypeptide is more soluble, i.e. better dissolves, than the parent polypeptide at that pH. Higher solubility results in easier formulation at high concentrations. The solubility of a polypeptide at a given pH can be determined by progressively increasing the protein content in the sample until saturation is reached. The protein content is then determined by quantification methods, such as SDS-PAGE, ELISA, BCA assay, Bradford assay, capillary electrophoresis, and ultraviolet absorbance; see also in the Examples.

Solubility can be fine-tuned by “resurfacing” of polypeptides, e.g. thermostable alpha-amylases. In enzyme resurfacing, surface-exposed and non-conserved residues are targeted to obtain better physico-chemical characteristics such as solubility, pH optimum, resistance to aggregation, and higher expressability (Chapman and McNaughton; Scratching the Surface; Resurfacing Proteins or Endow New Properties and Function, Cell Chem Biol (2016)).

“Higher resistance to protein aging includes resistance to oxidation. For example, a variant polypeptide is less oxidized over time in the presence of an oxidant than the parent polypeptide. Amino acids susceptible to oxidation include methionine. Oxidation of alpha-amylases reduces their specific activity, such that a higher resistance to oxidation prolongs the shelf-life, i.e. the duration for which the alpha-amylase retains sufficient specific activity. Similarly, higher resistance to oxidation results in the ability of the alpha amylase to be used in more oxidizing settings. Resistance to oxidation (and thereby to protein aging) can be assayed, e.g., by measuring the specific activity of an alpha-amylase over time when exposed to an oxidant, e.g. 0.1% H²O².

“Specific activity” means the activity of an enzyme per amount of that enzyme, typically per milligram. An increased specific activity means that, when equal amounts of the variant polypeptide and the parent polypeptide are used, more alpha-amylase activity will be exhibited by the amount of variant polypeptide than by the amount of parent polypeptide. Increased specific activity therefore allows for the use of less variant polypeptide to achieve a total activity compared to the parent polypeptide, reducing costs. The inventive variant polypeptides have increased specific activity by increasing the flexibility of their rotamers via amino acid modifications at amino acid positions 205 and 206 in the numbering of SEQ ID No. 1. The specific activity of a variant polypeptide can be determined by measuring alpha-amylase activity as described above and dividing the obtained total activity by the amount of variant polypeptide used, measured by protein quantification methods such as SDS-PAGE, ELISA, BCA assay, Bradford assay, capillary electrophoresis, and ultraviolet absorbance.

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as non-identical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

A sequence identity is determined by a program, which produces an alignment, and calculates identity counting both mismatches at a single position and gaps at a single position as non-identical positions in final sequence identity calculation. For example program Needle (EMBOS), which has implemented the algorithm of Needleman and Wunsch (Needleman and Wunsch, 1970, J. Mol. Biol. 48: 443-453), and which calculates sequence identity by first producing an alignment between a first sequence and a second sequence, then counting the number of identical positions over the length of the alignment, then dividing the number of identical residues by the length of an alignment, then multiplying this number by 100 to generate the % sequence identity [% sequence identity−(# of Identical residues/length of alignment)×100)].

A sequence identity can be calculated from a pairwise alignment showing both sequences over the full length, so showing the first sequence and the second sequence in their full length (“Global sequence identity”). For example, program Needle (EMBOSS) produces such alignments; % sequence identity−(# of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity−(# of Identical residues/length of alignment)×100)].

A sequence alignment is calculated wherein mismatches at a single position are counted as non-identical positions in final sequence identity calculation; however, gaps at a single position are not counted (ignored) as non-identical positions in final sequence identity calculation. The sequence alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open-10.0, gap extend-0.5 and matrix-EBLOSUM62). Then, a sequence identity can be calculated from the alignment showing both sequences over the full length, so showing the first sequence and the second sequence in their full length (“Global sequence identity”). For example: % sequence identity−(# of identical residues/length of alignment)×100)].

The variant polypeptides are described by reference to an amino acid sequence which is at least n % identical to the amino acid sequence of the respective parent enzyme with “n” being an integer between 80 and 100. The variant polypeptides include enzymes that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full length amino acid sequence of the parent alpha-amylase according to SEQ ID No. 1, wherein the variant polypeptide has alpha-amylase activity. The variant polypeptide also has an increased solubility at pH≤6.0, an increased solubility at pH≥10.0, a higher resistance to protein aging, and/or an increased specific activity compared to the parent polypeptide.

The variant polypeptide comprises a) at least one amino acid modification at an amino acid residue position number selected from the group consisting of: 23, 33, 125, 133, 157, 181, 214, 228, 260, 272, 323, 336, 349, 357, 407, and 408 or a combination thereof in the numbering of SEQ ID No. 1, or b) at least one amino acid modification at an amino acid residue position number selected from the group consisting of: 55 and 61 or a combination thereof in the numbering of SEQ ID No. 1, or c) amino acid modifications at amino acid residue position numbers 205 and 206 in the numbering of SEQ ID No. 1, or a combination thereof.

The term “amino acid modification” means that the amino acid sequence of the variant polypeptide is modified compared to the amino acid sequence of the parent polypeptide, i.e. the polypeptide according to SEQ ID No. 1 or SEQ ID No.2. The term “amino acid modification” is not intended to comprise modifications to an amino acid residue itself, such as, but not limited to, phosphorylation, myristoylation, palmitoylation, isoprenylation, acetylation, alkylation, amidation, gamma-carboxylation or glycoslation. The term “amino acid modification” includes amino acid substitution, amino acid insertion and amino acid deletion. Hence, the variant polypeptide of the present invention comprises at least one amino acid substitution, amino acid insertion and/or amino acid deletion compared to the parent polypeptide, i.e. the polypeptide according to SEQ ID No. 1 or SEQ ID No.2. Preferably, the amino acid modification is an amino acid substitution.

“Amino acid substitutions” are described by providing the original amino acid residue in the parent polypeptide followed by the number of the position of this amino acid residue within the amino acid sequence. For example, a substitution of amino acid residue 23 means that the amino acid of the parent at position 23 can be substituted with any of the 19 other amino acid residues and is designated as “23”. In addition, a substitution can be described by providing the original amino acid residue in the parent polypeptide. For example, the substitution of serine are residue 23 is designated as “Ser23” or “S23”. In addition, a substitution can be described by providing the original amino acid residue in the parent polypeptide followed by the number of the position of this amino acid residue within the amino acid sequence and followed by the specific substituted amino acid within the variant polypeptide. For example, the substitution of serine at position 23 with glutamate is designated as “Ser23Glu” or “S13E”. If more than one specific amino acid substitution follows the position number, e.g. “M55A/L/Q”, the parent amino acid (here: methionine) at the indicated position (here: position 55) can be substituted by any one of the listed substituted amino acids (here: either alanine, leucine, or glutamine). Combinations of substitutions are described by inserting comas between the amino acid residues, for example: S23E, N260E, Q272E, S407E represents a combination of substitutions of four different amino acid residues when compared to a parent polypeptide. Variants having a substitution on the amino acid level are encoded by a nucleic acid sequence which differs from the parent nucleic acid sequence encoding the parent polypeptide at least in the position encoding the substituted amino acid residue.

The amino acid substitution in the variant polypeptide may be a conservative amino acid substitution. A “conservative amino acid substitution” or “substitution with a related amino acid” means replacement of one amino acid residue in an amino acid sequence with a different amino acid residue having a similar property at the same position compared to the parent amino acid sequence. Some examples of a conservative amino acid substitution include, but are not limited to, replacing a positively charged amino acid residue with a different positively charged amino acid residue; replacing a polar amino acid residue with a different polar amino acid residue; replacing a non-polar amino acid residue with a different non-polar amino acid residue, replacing a basic amino acid residue with a different basic amino acid residue, or replacing an aromatic amino acid residue with a different aromatic amino acid residue.

A list of conservative amino acid substitutions is provided in the Table below (see for example Creighton (1984) Proteins. W.H. Freeman and Company (Eds)).

| Residue | Conservative Substitution(s) |
|---|---|
| Ala | Ser |
| Arg | Lys |
| Asn | Gln, His |
| Asp | Glu |
| Gln | Asn |
| Cys | Ser |
| Glu | Asp |
| Gly | Pro |
| His | Asn, Gln |
| Ile | Leu, Val |
| Leu | Ile, Val |
| Lys | Arg, Gln |
| Met | Leu, Ile |
| Phe | Met, Leu, Tyr |
| Ser | Thr, Gly |
| Thr | Ser, Val |
| Trp | Tyr |
| Tyr | Trp, Phe |
| Val | Ile, Leu |

An “amino acid insertion” is described by providing the number of the position within the amino acid sequence behind which the amino acid is inserted followed by an apostrophe and the specific inserted amino acid residue. For example, the insertion of serine behind position 132 is designated as “132'S”. Variants having an insertion on the amino acid level are encoded by a nucleic acid sequence which differs from the parent nucleic acid sequence encoding the parent polypeptide at least in the position encoding the inserted amino acid residue.

An “amino acid deletion” is described by providing the number of the position within the amino acid sequence at which the amino acid residue is deleted followed by a delta and the specific deleted amino acid residue. For example, the deletion of asparagine on position 125 is designated as “125ΔN”. Variants having deletions on the amino acid level are encoded by a nucleic acid sequence which differs from the parent nucleic acid sequence encoding the parent polypeptide at least at the position encoding the deleted amino acid residue.

In one embodiment, the variant polypeptide comprises a) at least one amino acid substitution selected from the group consisting of: S23E or M23E, Q33E, N125E or G125E, S133T, S157D, Q181E, N214D, N228S, N260D/E, Q272D, N323E, N336A, N349P, N357E, S407E and S408E or a combination thereof in the numbering of SEQ ID No. 1, preferably S407E, or b) at least one amino acid substitution selected from the group consisting of: M55A/L/Q and M61V or a combination thereof in the numbering of SEQ ID No. 1, preferably M55L, or c) amino acid modifications A205G and P206A in the numbering of SEQ ID No. 1. In a preferred embodiment, the variant polypeptide comprises the amino acid modifications of:
a) S23E and N125E, or
b) Q33E and S157D, or
c) Q181E and N260D, or
d) N214D and S407E, or
e) S23E, Q181E, N260D, Q272E, N323E, N349P, N357E, S407E, and S408D, or
f) S23E, Q181E, N228S, N260D, Q272E, N323E, N349P, N357E, and S408D, or
g) S23E, Q33E, Q181E, N260E, Q272D, N323E, N349P, N357E, and S407E, or
h) S23E, N260E, Q272E, and S407E, or
i) S23E, N214D, N260E, Q272E, and S407E, or
j) S23E, N214D, N260D, Q272E, and S407E, or
k) S23E, N260E, Q272E, N349P, and S407E, or
l) S23E, N260E, Q272E, and S407E, or
m) M55A and M61V, or
n) M55L and M61V, or
o) M55Q and M61V, or
p) A205G, P206A, and S407E, or
q) K9E, V12L, S23M, R31A, E37D, Y39A, D40S, M1021, N125G, K259N, S266D, Q269R, N270Y, E335D, V355A, D361S, G372A, A388A, Y404W, Y406D, S407E, and Y418H, or
r) K9E, V12L, S23M, R31A, E37D, Y39A, D40S, M1021, N125G, A205G, P205A, K259N, S266D, Q269R, N270Y, E335D, V355A, D361S, G372A, A388A, Y404W, Y406D, and Y418H in the numbering of SEQ ID No. 1.

The above variant polypeptides are characterized in that, when they comprise at least one amino acid modification at an amino acid residue position number selected from the group consisting of: 23, 33, 125, 133, 157, 181, 214, 228, 260, 272, 323, 334, 349, 357, 407, and 408 or a combination thereof in the numbering of SEQ ID No. 1, they have an increased solubility at pH≤6.0 compared to the polypeptide of SEQ ID No. 1 or SEQ ID No. 2.

The above variant polypeptides are further characterized in that, when they comprise at least one amino acid modification at an amino acid residue position number selected from the group consisting of: 23, 33, 125, 133, 157, 181, 214, 228, 260, 272, 323, 334, 349, 357, 407, and 408 or a combination thereof in the numbering of SEQ ID No. 1, they have an increased solubility at pH 10.0 compared to the polypeptide of SEQ ID No. 1 or SEQ ID No. 2.

The above variant polypeptides are further characterized in that, when they comprise at least one amino acid modification at an amino acid residue position number selected from the group consisting of: 55 and 61 or a combination thereof in the numbering of SEQ ID No. 1, they are more resistant to protein aging compared to the polypeptide of SEQ ID No. 1 or SEQ ID No. 2. Preferably, the above variant polypeptides are further characterized in that, when they comprise at least one amino acid modification at an amino acid residue position number selected from the group consisting of: 55 and 61 or a combination thereof in the numbering of SEQ ID No. 1, they are more resistant to protein aging compared to the polypeptide of SEQ ID No. 1 or SEQ ID No. 2

The above variant polypeptides are further characterized in that, when they comprise amino acid modifications at amino acid residue position numbers 205 and 206 in the numbering of SEQ ID No. 1, they have increased specific activity compared to the polypeptide of SEQ ID No. 1 or SEQ ID No. 2.

The variant polypeptide may be a fragment. A “fragment” of an alpha-amylase is understood to refer to a smaller part of the alpha-amylase which consists of a contiguous amino acid sequence found in the amino acid sequence of the alpha-amylase and which has alpha-amylase activity. The skilled person knows that for a fragment to be enzymatically active the fragment has to comprise at least the amino acids present in the catalytic center of the alpha-amylase. These amino acids are either known for a given alpha-amylase or can easily be identified by the skilled person, for example by homology screening or mutagenesis. Further the fragment must comprise the indicated modified residues. Preferably, the fragment of the alpha-amylase has an increased solubility at pH≤6.0 or pH≥10.0, a higher resistance to protein aging, and/or an increased specificity compared to the full-length polypeptide according to SEQ ID No. 1 or SEQ ID No. 2 Preferably, the fragment comprises at least 70%, at least 80%, at least 85%, at least 90%, at least 95%, at least 96%, at least 97%, at least 98%, or at least 99% of the amino acids of the full-length polypeptide according to SEQ ID No.1 or SEQ ID No. 2.

The variant polypeptide may comprise a hybrid of at least one variant polypeptide and a second polypeptide having amylase activity, wherein the hybrid has alpha-amylase activity. For example, the variant polypeptide having alpha-amylase activity may be a hybrid of more than one alpha-amylase enzyme. A “hybrid” or “chimeric” or “fusion protein” means that a domain of a first variant polypeptide alpha-amylase is combined with a domain of a second alpha-amylase to form a hybrid amylase and the hybrid has alpha-amylase activity. Preferably, the hybrid alpha-amylase has an increased solubility at pH≤6.0 or pH≥10.0, a higher resistance to protein aging, and/or an increased specificity compared to the polypeptide according to SEQ ID No. 1 or SEQ ID No. 2. A domain of variant polypeptides having alpha-amylase enzyme activity can be combined with a domain of a commercially available amylase, such as Veron® available from AB Enzymes; Validase BAA, BakeDream®, BakeZyme™, and Panamore® available from DSM; POWERSoft®, Max-LIFEM, POWERFlex®, and POWERFresh® available from DuPont; BAN™, Liquozyme® SCDC, Natalase®, Stainzyme® plus, Fungamyl®, Novamyl®, OptiCake®, and Sensea® available from Novozymes; Amzyme TX available from Parchem; Aquazym 1201, Aquazym Ultra 2501, and Thermamyl®, Takaterm available from Novo Nordisk; Enzymex (Cocktail) available from Exotic Biosolutions Pvt. Ltd.; and Fructamyle FHT available from ERBSLOEH. In addition, domains from various amylase enzymes can be recombined into a single enzyme, wherein the enzyme has alpha-amylase activity. Preferably, the hybrid alpha-amylase comprising domains from various amylase enzymes has an increased solubility at pH≤6.0 or pH≥10.0, a higher resistance to protein aging, and/or an increased specificity compared to the polypeptide according to SEQ ID No. 1 or SEQ ID No. 2.

The variant polypeptides having alpha-amylase activity may be a “mature polypeptide.” A mature polypeptide means an enzyme in its final form including any post-translational modifications, glycosylation, phosphorylation, truncation, N-terminal modifications, C-terminal modifications or signal sequence deletions. A mature polypeptide can vary depending upon the expression system, vector, promoter, and/or production process.

“Enzymatic activity” means at least one catalytic effect exerted by an enzyme. Enzymatic activity is expressed as units per milligram of enzyme (specific activity) or molecules of substrate transformed per minute per molecule of enzyme (molecular activity). Enzymatic activity can be specified by the enzymes actual function and within the present invention means alpha-amylase activity as described above.

Enzymatic activity changes during storage or operational use of the enzyme. The term “enzyme stability” relates to the retention of enzymatic activity as a function of time during storage or operation.

To determine and quantify changes in catalytic activity of enzymes stored or used under certain conditions over time, the “initial enzymatic activity” is measured under defined conditions at time zero (100%) and at a certain point in time later (x %). By comparison of the values measured, a potential loss of enzymatic activity can be determined in its extent. The extent of enzymatic activity loss determines the stability or non-stability of an enzyme.

Parameters influencing the enzymatic activity of an enzyme and/or storage stability and/or operational stability are for example pH, temperature, and presence of oxidative substances.

“pH stability”, refers to the ability of a protein to function over a specific pH range. In general, most enzymes are working under conditions with rather high or rather low pH ranges.

The variant polypeptide may be active over a broad pH at any single point within the range from about pH 4.0 to about pH 12.0. The variant polypeptide having alpha-amylase activity is active over a range of pH 4.0 to pH 11.0, pH 4.0 to pH 10.0, pH 4.0 to pH 9.0, pH 4.0 to pH 8.0, pH 4.0 to pH 7.0, pH 4.0 to pH 6.0, or pH 4.0 to pH 5.0. The variant polypeptide having alpha-amylase enzyme activity is active at pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6 pH 8.7, pH 8.8 pH 8.9, pH 9.0, pH 9.1, pH 9.2, pH 9.3, pH 9.4, pH 9.5, pH 9.6, pH 9.7, pH 9.8, pH 9.9, pH 10.0, pH 10.1, pH 10.2, pH 10.3, pH 10.4, pH 10.5, pH 10.6, pH 10.7, pH 10.8, pH 10.9, pH 11.0, pH 11.1, pH 11.2, pH 11.3, pH 11.4, pH 11.5, pH 11.6, pH 11.7, pH 11.8, pH 11.9, pH 12.0, pH 12.1, pH 12.2, pH 12.3, pH 12.4, and pH 12.5, pH 12.6, pH 12.7, pH 12.8, pH 12.9, and higher.

Variant polypeptides may be active over a broad temperature range, wherein the temperature is any point in the range from about 20° C. to about 60° C. The variant polypeptides having alpha-amylase enzyme activity are active at a temperature range from 20° C. to 55° C., 20° C. to 50° C., 20° C. to 45° C., 20° C. to 40° C., 20° C. to 35° C., 20° C. to 30° C., or 20° C. to 25° C. Preferably, the variant polypeptides having alpha-amylase enzyme activity are active at a temperature of at least 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37 C, 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 6r C, 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., or higher temperatures.

The variant polypeptides having alpha-amylase enzyme activity may be used formulated alone or as a mixture of enzymes.

The formulation containing the variant polypeptide of the present invention may be a solid form such as powder, a lyophilized preparation, a granule, a tablet, a bar, a crystal, a capsule, a pill, a pellet, or in a liquid form such as in an aqueous solution, an aerosol, a gel, a paste, a slurry, an aqueous/oil emulsion, a cream, a capsule, or in a vesicular or micellar suspension.

The variant polypeptide of the present invention may be used in combination with at least one other enzyme. The other enzyme may be from the same class of enzymes, for example, may be a second alpha-amylase. The other enzyme may also be from a different class of enzymes, for example may be a lipase. The combination with at least one other enzyme may be a composition comprising at least three enzymes. The three enzymes may be from the same class of enzymes, for example the combination may comprise the variant polypeptide of the present invention, a second amylase, and a third amylase; or the enzymes may be from different class of enzymes for example the combination may comprise the variant polypeptide of the present invention, a lipase, and a xylanase.

The second enzyme may be selected from the group consisting of: a second alpha-amylase, a beta-amylase, a glucan 1, 4-alpha-maltotetraohydrolase, also known as exo-maltotetraohydrolase, G4-amylase; a glucan 1,4-alpha-maltohydrolase, also known as maltogenic alpha-amylase, a cyclodextrin glucanotransferase, a glucoamylase; an endo-1,4-beta-xylanase; a xylanase, a cellulase, an oxidoreductase; a phospholipase A1; a phospholipase A2; a phospholipase C; a phospholipase D; a galactolipase, a triacylglycerol lipase, an arabinofuranosidase, a transglutaminase, a pectinase, a pectate lyase, a protease, or any combination thereof. The enzyme combination may comprise the variant polypeptide of the present invention and a lipase, or the enzyme combination may comprise the variant polypeptide of the present invention, a lipase, and a xylanase.

The present invention is also directed to a composition comprising the variant polypeptide of the present invention.

The composition comprising the variant polypeptide of the present invention may also comprise a second enzyme.

Preferably the second enzyme is selected from the group consisting of: a second alpha-amylase, a lipase, a beta-amylase, a G4-amylase, a xylanase, a protease, a cellulase, a glucoamylase, an oxidoreductase, a phospholipase, and a cyclodextrin glucanotransferase.

In an aspect, the present invention provides a method of making a variant polypeptide comprising: providing a template nucleic acid sequence encoding the variant polypeptide, transforming the template nucleic acid sequence into an expression host, cultivating the expression host to produce the variant polypeptide, and purifying the variant polypeptide.

Preferably, the variant alpha-amylase according to the present invention is a recombinant protein which is produced using bacterial, fungal, yeast, or synthetic expression systems. “Expression system” also means a host microorganism, expression hosts, host cell, production organism, or production strain and each of these terms can be used interchangeably. Examples of expression systems include, but are not limited to: Aspergillus niger, Aspergillus oryzae, Hansenula polymorpha, Thermomyces lanuginosus, Fusarlum oxysporum, Fusarlum heterosporum, Escherlchla coll, Bacillus, preferably Bacillus subtilis or Bacillus licheniformis, Pseudomonas, preferably Pseudomonas fluorescens, Pichia pastoris (also known as Komagataella phaffi), Mycellopthora thermophila (C1), Schizosaccharomyces pombe, Trichoderma, preferably Trichoderma reesei and Saccharomyces, preferably Saccharomyces cerevisiae.

In a preferred embodiment, the bacterial expression system is selected from an E col, a Bacillus, a Pseudomonas, and a Streptomyces. In another preferred embodiment, the yeast expression system is selected from a Candida, a Komagataella, a Saccharomyces, a Schizosaccharomyces. In another preferred embodiment, the fungal expression system is selected from a Penicillium, an Aspergillus, a Fusarlum, a Myceliopthora, a Rhizomucor, a Rhizopus, a Thermomyces, and a Trichoderma.

“Transforming” means the introduction of exogenous DNA into an expression host by methods well known to the person skilled in the art.

“Purifying” means the removal of other cellular material of the expression host from the variant polypeptide by methods well established in the art.

The variant polypeptide of the present invention may be useful for industrial applications. The variant polypeptide having alpha-amylase enzyme activity may be used in a detergent, a personal care product, in the processing of textiles, in pulp and paper processing, in the production of ethanol, lignocellulosic ethanol, or syrups; or as viscosity breakers in oilfield and mining industries.

In an aspect, the variant polypeptide is used for processing starch. Preferably, the starch is processed to fructose.

In an aspect, the variant polypeptide is used for cleaning or washing textiles, hard surfaces, or dishes.

In an aspect, the variant polypeptide is used for making ethanol.

In an aspect, the variant polypeptide is used for treating an oil well.

In an aspect, the variant polypeptide is used for processing pulp or paper.

In an aspect, the variant polypeptide is used for animal feed.

In an aspect, the variant polypeptide is used for syrup production.

In an aspect, the variant polypeptide is used for preparing a dough or a baked product prepared from the dough.

“Dough” is defined as a mixture of flour, salt, yeast and water, which may be kneaded, molded, shaped or rolled prior to baking. In addition, also other ingredients such as sugar, margarine, egg, milk, etc. might be used. The term includes doughs used for the preparation of baked goods, such as bread, rolls, sandwich bread, baguette, ciabatta, croissants, sweet yeast doughs, etc.

The term “baked products” includes, but is not limited to, baked products such as bread, crispy rolls, sandwich bread, buns, baguette, ciabatta, croissants, noodles, as well as fine bakery wares like donuts, brioche, stollen, cakes, muffins, etc. Baked products include, but are not limited to: bread, rolls, buns, pastries, cakes, flatbreads, pizza bread, pita bread, wafers, pie crusts, naan, lavish, pitta, focaccia, sourdoughs, noodles, cookies, doughnuts, deep-fried tortillas, pancakes, crepes, croutons, and biscuits. The baked product could also be an edible container such as a cup or a cone.

Baking bread generally involves mixing ingredients to form a dough, kneading, rising, shaping, baking, cooling and storage. The ingredients used for making the dough generally include flour, water, salt, yeast, and other food additives. The variant polypeptide of the present invention for use in preparing a dough or a baked product prepared from the dough is one of the ingredients used for making the dough.

Flour is generally made from wheat and may be milled for different purposes such as making bread, pastries, cakes, biscuits pasta, and noodles. Alternatives to wheat flour include, but are not limited to: almond flour, coconut flour, chia flour, corn flour, barley flour, spelt flour, soya flour, hemp flour, potato flour, quinoa, teff flour, rye flour, amaranth flour, arrowroot flour, chick pea (garbanzo) flour, cashew flour, flax meal, macadamia flour, millet flour, sorghum flour, rice flour, tapioca flour, and any combination thereof. Flour type is known to vary between different regions and different countries around the world.

Treatment of flour or dough may include adding inorganic substances, organic substances such as fatty acids, carbohydrates, amino acids, proteins, and nuts. The flour or dough may be pretreated prior to baking by cooling, heating, irradiation, agglomeration, or freeze-drying. In addition, the flour or dough may be pretreated prior to baking by adding enzymes such as the variant polypeptide of the present invention, or micro-organisms, such as yeasts.

Yeast breaks down sugars into carbon dioxide and water. A variety of Baker's yeast, which are usually derived from Saccharomyces cerevisiae, are known to those skilled in the art including, but not limited to: cream yeast, compressed yeast, cake yeast, active dry yeast, instant yeast, osmotolerant yeasts, rapid-rise yeast, deactivated yeast. Other kinds of yeast include nutritional yeast, brewer's yeast, distiller's and wine yeast.

Sweeteners which can be added to the dough include, but are not limited to: liquid sugar, syrups, white (granulated) sugars, brown (raw) sugars, honey, fructose, dextrose, glucose, high fructose corn syrup, molasses, stevia and artificial sweeteners.

Emulsifiers which can be added to the dough include, but are not limited to, diacetyl tartaric acid esters of monoglycerides (DATEM), sodium stearoyl lactylate (SSL), calcium stearoyl lactylate (CSL), ethoxylated mono- and diglycerides (EMG), polysorbates (PS), and succinylated monoglycerides (SMG).

Other food additives which may be used in the methods of baking include: lipids, oils, butter, margarine, shortening, butterfat, glycerol, eggs, diary, non-diary alternatives, thickeners, preservatives, colorants, and enzymes.

Ingredients or additives for baking may be added individually to the dough during the baking process. The ingredients or additives may also be combined with more than one ingredient or additive to form pre-mixes and then the pre-mixes are added to the dough during the baking process. The flour or dough mixtures may be prepared prior to baking including ready-for oven doughs, packaged doughs or packaged batters.

Bakery products may be modified to meet special dietary requirements such as sugar-free diet, gluten-free diet, low fat diet, or any combination thereof. The enzymes may extend shelf-life of a dough-based product or provide antimicrobial (mold-free) effects.

“Bread volume” is the volume of a baked good determined by using a laser scanner (e.g. Volscan Profiler ex Micro Stable System) to measure the volume as well as the specific volume. The term also includes the volume which is determined by measuring the length, the width and the height of certain baked goods.

The use of the variant polypeptide of the present invention in a method of making a dough increases the resilience of the baked product prepared from the dough. The baked product may be stored for five days, 10 days, 15 days or 20 days, before resilience is determined. The resilience can be determined by a texture analyzer test using the Texture Profile Analysis (TPA). The TPA is a two cycle compression test and the resilience is calculated by Recoverable work done divided by hardness work done by the texture analyzer. The resilience of a baked product prepared from dough using the variant polypeptide of the present invention is increased by at least 5% or 8%, preferably by at least 10% or 12%, more preferably by at least 15% or 20% and most preferably by at least 25% or 30%.

The use of the variant polypeptide of the present invention in a method of making a dough decreases the hardness of the baked product prepared from the dough after storage. Typically, the baked product is stored for 10 days, 15 days or 20 days at room temperature, before the hardness is determined. The hardness may be determined according to the AACC 74-09 test, for example using a 35 mm sample and 5 kg load cell. The following parameters may be used in the test: Pre-test speed: 1 mm/sec, Test speed: 5 mm/sec, Post-Test speed: 5 mm/sec, Target Mode: Distance, Distance: 10 mm, Time 5 sec, Trigger Type: Auto (Force), Trigger Force: 5 g. The hardness of a baked product prepared from dough using the variant polypeptide of the present invention is decreased by at least 5% or 8%, preferably by at least 10% or 12%, more preferably by at least 15% or 20%, still more preferably by at least 25% or 30%, and most preferably by at least 35 or 40%.

In an aspect, the variant polypeptide is used in a detergent or personal care product.

Since detergents have mainly alkaline pH values, α-amylases that are active in alkaline environments (e.g. at pH 10 or above) are especially preferred in this context.

A detergent or personal care product may comprise from 0.000001 percent by weight to 5% by weight, in particular from 0.00001 to 3% by weight, of the variant polypeptide, and may additionally include other enzymes, in particular hydrolytic enzymes or oxidoreductases, particularly preferably further amylases, proteases, lipases, cutinases, hemicellulases, cellulases, 6-glucanases, oxidases, peroxidases, perhydrolases and/or laccases. The detergent or personal care product may be overall solid, preferably after a compacting step for at least one of the included components, particularly preferably that it is overall compacted; or it may be overall liquid, gel-like or paste-like, preferably with encapsulation of at least one of the included components, particularly preferably with encapsulation of at least one of the included enzymes, very particularly preferably with encapsulation of the variant polypeptide.

As active components in detergents, the variant polypeptides may be used for cleaning textiles or solid surfaces, such as, for example, crockery, floors or utensils. In these applications, the amylolytic activity serves to break down by hydrolysis, or detach from the substrate, carbohydrate-containing contaminations and in particular those based on starch. In this connection, they may be used alone, in suitable media or else in detergents. The conditions to be chosen for this, such as, for example, temperature, pH, ionic strength, redox conditions or mechanical effects, should be optimized for the particular cleaning problem, i.e. in relation to the soiling and the substrate. Thus, usual temperatures for detergents are in ranges from 10° C., for manual compositions via 40° C. and 60° C., up to 95° C. for machine compositions or for industrial applications. Since the temperature can usually be adjusted continuously in modern washing and dishwashing machines, all intermediate temperatures are also included. The ingredients of the relevant agents are preferably also matched to one another. The other conditions can likewise be designed very specifically for the particular cleaning purpose via the other components of said agents.

Preferred detergents are distinguished by the washing or cleaning performance of the agent in question being improved by adding the variant polypeptide of the invention, compared with the formulation without this variant polypeptide. Preference is given to synergies with respect to cleaning performance.

A variant polypeptide of the invention can be used both in compositions for large-scale consumers or industrial users and in products for the private consumer. The detergents of the invention thus mean any conceivable types of cleaning compositions, both concentrates and compositions to be applied in an undiluted form; for use on a commercial scale, in the washing machine or for washing or cleaning by hand. They include, for example, detergents for textiles, carpets or natural fibers, for which agents the term detergent is used according to the present invention. They include also, for example, dishwashing agents for dishwashers or manual washing-up liquids or cleaners for hard surfaces such as metal, glass, porcelain, ceramics, tiles, stone, painted surfaces, plastics, wood or leather; for these, the term cleanser is used according to the present invention.

Detergents and personal care products include, for example, solids, pulverulent, liquid, gel-like or paste-like compositions, where appropriate also composed of two or more phases, compressed or uncompressed; they also include for example: extrudates, granules, tablets or pouches, packaged both in large containers and in portions.

The variant polypeptide may be combined with one or more of the following ingredients: nonionic, anionic and/or cationic surfactants, bleaches, bleach activators, bleach catalysts, builders and/or cobuilders, solvents, thickeners, sequestering agents, electrolytes, optical brighteners, antiredeposition agents, corrosion inhibitors, in particular silver protectants, soil release agents, color transfer inhibitors, foam inhibitors, abrasives, dyes, fragrances, antimicrobial agents, UV stabilizers, enzymes such as, for example, proteases, (where appropriate other) amylases, lipases, cellulases, hemicellulases or oxidases, stabilizers, in particular enzyme stabilizers, and other components known in the art.

The following examples are provided for illustrative purposes. It is thus understood that the examples are not to be construed as limiting. The skilled person will clearly be able to envisage further modifications of the principles laid out herein.

### EXAMPLES

Unless otherwise indicated, all Examples use the processes as laid out in Example 1.

### Example 1: General Methods

### 1. Expression

Parent or variant alpha-amylases were expressed in Pseudomonas fluorescens. A strain not expressing either amylase served as a control. Bacterial culture pellets were stored at −20° C.

### 2. Pellet Recovery

Frozen pellets from 1. were thawed, resuspended in resuspension buffer (10 mM CaCl2, 2 mM MgSO4, 1% glycerol) and incubated with Benzonase® Nuclease (E1014, Millipore Sigma) and centrifuged. Supernatants and pellets were both harvested and stored at 4° C.

### 3. Formulation

Supernatant from 2. Was combined with glycerol, the pH adjusted to 6.5 and the concentration adjusted to the target concentration with water.

### 4. PAHBAH Assay

Quantitation of starch hydrolysis for the alpha-amylase and variant enzymes was measured using the 4-Hydroxybenzhydrazide method as described in Lever M. (1972) Anal. Biochem. 47, 273-279, with the following modifications. After reaction of enzyme with 1% cornstarch at 80° C., samples were taken at 20 seconds, 1, 2, 3, 4, 6, 8, and 10 minutes, quenched with 1% PAHBAH reagent, boiled, and the solution absorption was read at 410 nm in a BioTek plate reader.

### 5. Determination of Solubility

10 mL of stock solution of parent or variant alpha-amylases (from 2. or 3.) were transferred into a Falcon tube, V 4.1 M NH₄SO₄ was added and tubes incubated at 4° C. Solutions were centrifuged at 4500 g. The supernatants were decanted and stored at 4° C. Pellets were resuspended in 0.24 M NH₄SO₄, and centrifugation repeated. Pellets were resuspended in 20 mM CAPS buffer pH 10. Solution was dialyzed at 4 C overnight in 20 mM CAPS pH 10. Half of the total volume from the dialysis cassette was transferred to an Eppendorf tube and dialyzed at 4° C. overnight in 20 mM MES buffer at pH 6. Each solution was concentrated using 0.5 mL or 2 mL 10-kDa MWCO concentrators at 20° C. Samples were centrifuged at 30,000 rpm for 10 min at room temperature. Supernatants were diluted 20-fold with pH 6 or pH 10 buffer. A280 (with A340 correction) was measured by nanodrop and concentrations were calculated using ε=195,735 M⁻¹ cm⁻¹ and MW=49,639 Da.

### 6. Oxidation LCMS Analysis

Sample Preparation: Sample in pH 10 CAPS was added to a new centrifuge tube and urea was added. 0.5 M TCEP was added and resulting mixture was heated for 10 minutes at 60° C. Solution was then cooled to RT and 0.5 M iodoacetamide was added. This solution was incubated in the dark for 30 minutes. For the control, pH 10 CAPS was diluted with water and then processed in the same manner as other samples. Following carbamidomethylation, 50 mM pH 8.0 TRIS-HCl was added to each tube along with solution of trypsin/LysC. Samples were incubated overnight at 37° C. Resulting digests were purified, speed vacuumed to dryness and resuspended in 5% acetonitrile in water with 0.1% formic acid.

LCMS:Separation was achieved on an Agilent 1290 infinity UPLC using a Waters ACQUITY UPLC BEH C18, 130 Å, 1.7 μm, 1 mm×100 mm column with 90-minute gradient elution. Mobile phase A was comprised of 5% acetonitrile in water with 0.1% formic acid while mobile phase B was comprised of 0.1% formic acid in acetonitrile. The column compartment was held at 45° C. and a constant flow rate of 50 μL/min was used. Sample injection volumes were 5 μL. Eluted peptides were electrosprayed into a SCIEX 5600 QTOF using the following source parameters: source voltage of 5 kV, heater at 500 C, ion source gas at 35, ion source gas 2 at 35, Curtain Gas 20. Full MS and IDA (MS/MS) experiments were acquired. IDA experiments were collected using dynamic accumulation and a former ion exclusion window of 30 s.

### Example 2: Generation of Variant Alpha-Amylase Enzymes to Improve Solubility at Low pH

The alpha-amylase according to SEQ ID No. 1 is a highly thermostable alpha-amylase. Many of the applications described elsewhere herein require activity at low pH. Despite the activity-based pH profile of this alpha-amylase having a maximum around pH 5.5, the enzyme solubility is poor at that pH, as it is close to its pl of 4.51.

To address the solubility issue, resurfacing was used. The design of the different variants accounted for both sequence and structural information of the enzyme. First, the conservation scores were determined and conserved residues. Then, structural information was obtained by generation of a homology model. The solvent exposed surface accessible area (SASA) of each amino acid was calculated to distinguish among the buried and exposed amino acids of the enzyme. Further, the electrostatic potential map for the enzyme was calculated. This allowed the adjustment of protein solubility by alteration of surface-exposed amino acids with positively or negatively charged amino acids. Care was also taken not to mutate the functional amino acids and also amino acids surrounding the substrate binding site. Amino acids with the characteristics of being surface-exposed, non-functional, and non-conserved were selected. Those were modelled as changed to achieve better packing, avoid steric clash, increase salt bridge formation and increase the surface charge. Finally, a list of 16 single residue variants and 12 multiple residue variants was proposed. Genetic constructs encoding for the proposed variants were created, integrated into expression vectors, and these expression vectors were transformed into Pseudomonas fluorescens for further experimental validation. Table 1 lists the generated variants.

TABLE 1
Alpha-amylase variants designed to improve solubility at low pH

| Sample Name | Amino acid modifications |
|---|---|
| Variant 1 | S23E |
| Variant 2 | Q33E |
| Variant 3 | N125E |
| Variant 4 | S133T |
| Variant 5 | S157D |
| Variant 6 | Q181E |
| Variant 7 | N214D |
| Variant 8 | N228S |
| Variant 9 | N260D |
| Variant 10 | Q272D |
| Variant 11 | N323E |
| Variant 12 | N357E |
| Variant 13 | N336A |
| Variant 14 | N349P |
| Variant 15 | S407E |
| Variant 16 | S408E |
| Variant 17 | S23E, N125E |
| Variant 18 | Q33E, S157D |
| Variant 19 | Q181E, N260D |
| Variant 20 | N214D, S407E |
| Variant 21 | S23E, Q181E, N260D, Q272E, N323E, |
| | N357E, N349P, S407E, S408D |
| Variant 22 | S23E, Q181E, N228S, N260D, Q272E, |
| | N323E, N357E, N349P, S408D |
| Variant 23 | S23E, Q33E, Q181E, N260E, Q272D, |
| | N323E, N357E, N349P, S407E |
| Variant 24 | S32E, N260E, Q272E, S407E |
| Variant 25 | S32E, N214D, Q272E, N260E, S407E |
| Variant 26 | S32E, N214D, Q272E, N260D, S407E |
| Variant 27 | S23E, N260E, Q272E, N349P, S407E |
| Variant 28 | S23E, N260E, Q272E, S407E |

While no residue is in direct proximity of the substrate binding cleft, most of the sites belong to flexible loops, with the exceptions of Q33, N214, N336, and N349 being part of secondary structures. The majority of substitutions are targeting the surface by replacing polar residues with anionic amino acids—the intended outcome is shifting the pl of the alpha-amylase according to SEQ ID No. 1 to lower values, as protein solubility is at its lowest when approaching the pl. Finally, S133T, N228S, N336A, and N349P are neutral mutations. Specifically, S133T and N349P seek to reduce the flexibility of two loops (Huang and Nau: A Conformational Flexibility Scale for Amino Acids in Peptides; Angewandte Chemie International Edition (2003)), with N349P intercalating the coordinating triad for a Ca²⁺-site.

### Example 3: Solubility Testing of Variant Alpha-Amylase Enzymes Generated to Improve Solubility at Low pH

Variants 1-28 were expressed according to Example 1 in shake flasks, and recovered.

After consideration of the biochemical characterization outcomes, variants 1, 2, 3, 11, 12, 13, 14, 15, 16, and 27 were selected to be moved forward for confirmation of the results. In addition, half of the recovered volumes were formulated in sodium citrate and glycerol, aiming for a final pH of 6.5 and a glycerol content of ˜50%.

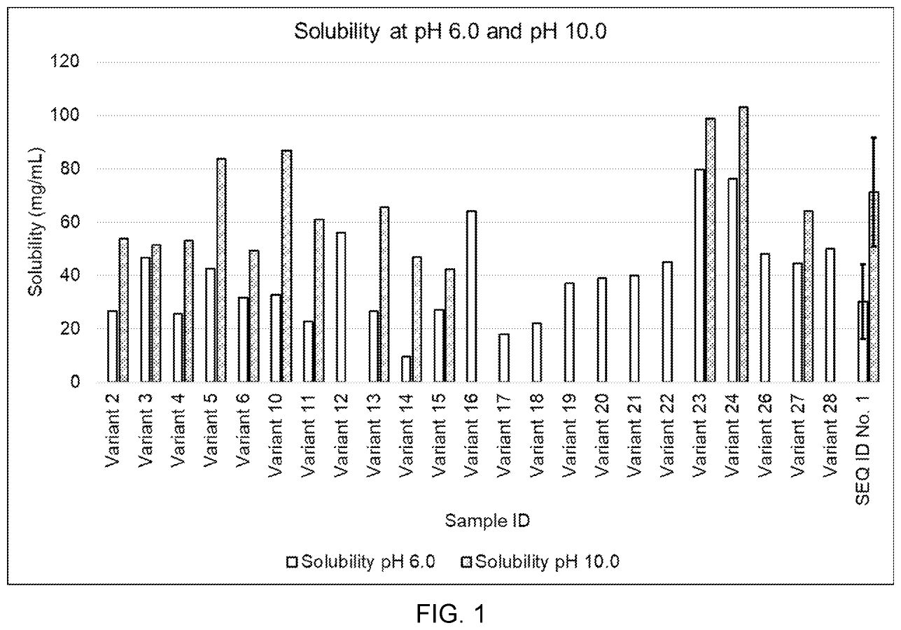

Traditionally, measuring protein solubility is hampered by the challenge of developing quantitative solubility assays. Solubility is defined as the concentration at which a solution is saturated with a chosen analyte, and it is thus representative of a thermodynamic equilibrium between its soluble and insoluble states in a given set of conditions. As such, solubility is meant to be measured once saturation is reached—which can be difficult for proteins prone to aggregation. In those cases, solubility measurements can be obtained by artificially lowering the solubility of the protein with the use of an additive. A classical reagent for protein precipitation is ammonium sulfate. Ammonium sulfate acts as a kosmotropic salt, outcompeting water in the solvation layer and triggering self-association. The solubility at pH 6.0 and 10.0 of the resurfacing variants were then obtained by NH₄SO₄ precipitation and resuspension in the appropriate buffer, generating saturated solution of variants at the pH of interest. The concentration of the protein in the supernatant was then measured by absorbance at 280 nm (see FIG. 1). Overall, Variant 23 and 24, which are both multiple residue variants, stood out in terms of solubility at both pH 6.0 and 10.0, with an outstanding ˜80 mg/mL at pH 6.0. Among single mutants, Variant 12 and Variant 16 showed an improvement in solubility of up to 2.2-fold compared to the alpha-amylase according to SEQ ID No. 1.

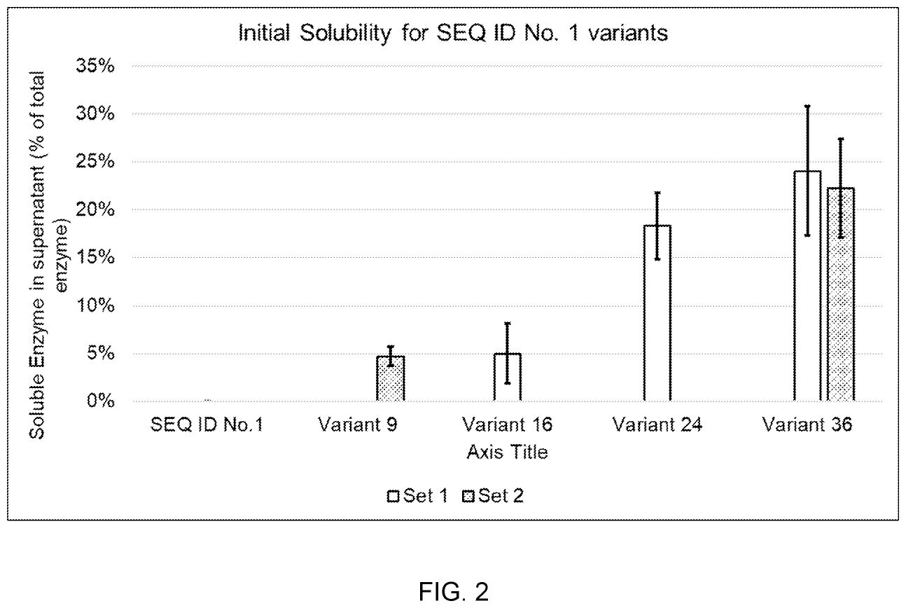

Finally, initial solubility, solubilization at high pH, Solubility at pH 10.0 and solubility at pH 6.0 were tested for variants 9, 16, and 24 as well as variant 36 (corresponding to SEQ ID No.1 with the following amino acid modifications: K9E, V12L, S23M, R31A, E37D, Y39A, D40S, M1021, N125G, K259N, S266D, Q269R, N270Y, E335D, V355A, D361S, G372A, A388A, Y404W, Y406D, S407E, and Y418H and corresponding to SEQ ID No. 2 with the amino acid modification S407E) as laid out in Example 1. Results are shown in FIG. 2. Table 2 ranks the performance of the variants and the alpha-amylase according to SEQ ID No.1.

TABLE 2
Rankings of Solubilization and Solubility Performance

| | Initial | Solubilization | Solubility | Solubility |
|---|---|---|---|---|
| Alpha-amylase | solubility | at high pH | at pH 10.0 | at pH 6.0 |
| SEQ ID No. 1 | 5 | 5 | — | — |
| Variant 9 | 3 | 2 | 3 | 4 |
| Variant 16 | 3 | 2 | 2 | 1 |
| Variant 24 | 2 | 2 | 4 | 3 |
| Variant 36 | 1 | 1 | 1 | 2 |

### Example 4: Generation and Testing of Variants Designed to Increase Resistance to Protein Aging

The alpha-amylase according to SEQ ID No. 1 loses specific activity when oxidized. Top residues impacted by oxidation as determined by mass spectrometry were M55 and M102. Further, residue M61 was found to spontaneously oxidize in freshly produced samples of the alpha-amylase corresponding to SEQ ID No. 1. Variants including substitutions of residues M55 and M61 were generated and expressed and purified as described in Example 1. These variants are described in Table 3.

TABLE 3
Alpha-amylase variants designed to
improve resistance to protein aging

| Sample Name | Amino acid modifications |
|---|---|
| Variant 29 | M55A |
| Variant 30 | M55L |
| Variant 31 | M55Q |
| Variant 32 | M61V |
| Variant 33 | M55A, M61C |
| Variant 34 | M55L, M61V |
| Variant 35 | M55Q, M61V |

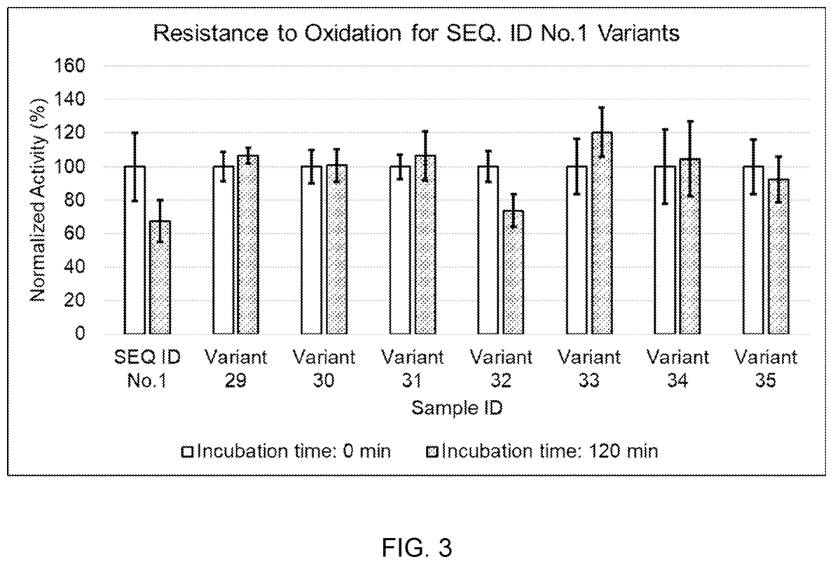

Variants 29-35 as well as variant 36 from Example 3 were tested for specific activity upon oxidization as described in Example 1. Variants 29-35 showed resistance to oxidation, i.e. did not lose specific activity (see FIG. 3). The alpha-amylases according to SEQ ID No.1 and SEQ ID No. 2 as well as variant 36, all of which do not include amino acid modification for resisting oxidation, i.e. protein aging, were not resistant to oxidation, i.e. showed decreased specific activity after oxidation (see FIG. 3).

### Example 5: Generation and Testing of Variants Designed to Improve Specific Activity

To improve the specific activity of the alpha amylase according to SEQ ID No. 1 or 2, variants with amino acid modifications at these residues were generated, expressed and purified as described in Example 1. These variants are described in Table 4.

TABLE 4
Alpha-amylase variants designed to improve specific activity

| Sample Name | Amino acid modifications |
|---|---|
| Variant 41 | A205G, P206A, S407E |
| Variant 40 | K9E, V12L, S23M, R31A, E37D, Y39A, |
| | D40S, M102I, N125G, A205G, P205A, |
| | K259N, S266D, Q269R, N270Y, E335D, |
| | V355A, D361S, G372A, A388A, Y404W, |
| | Y406D, Y418H |

Variant 41 also includes an amino acid modification that improves solubility at pH 6.0 and pH 10.0. Variant 40 corresponds to the alpha-amylase of SEQ ID No. 2 with the amino acid modification A205G and P206A.

1-28. (canceled)

29. A variant polypeptide of alpha-amylase according to SEQ ID NO:1 having alpha-amylase activity and comprising an amino acid sequence which is at least 80% identical to the sequence according to SEQ ID NO:1, wherein the amino acid sequence comprises
a) at least one amino acid modification at an amino acid residue position number selected from the group consisting of: 23, 33, 125, 133, 157, 181, 214, 228, 260, 272, 323, 336, 349, 357, 407, and 408 or a combination thereof in the numbering of SEQ ID NO:1, or
b) at least one amino acid modification at an amino acid residue position number selected from the group consisting of: 55 and 61 or a combination thereof in the numbering of SEQ ID NO:1, or
c) amino acid modifications at amino acid residue position numbers 205 and 206 in the numbering of SEQ ID NO:1,
or a combination thereof.

30. The variant polypeptide of claim 29, wherein the amino acid modification comprises an amino acid substitution, insertion, deletion, or a combination thereof.

31. The variant polypeptide of claim 30, wherein the amino acid modification comprises an amino acid substitution, and wherein the amino acid substitution is a conservative amino acid substitution.

32. The variant polypeptide of claim 30, wherein the amino acid substitution comprises S23E, M23E, Q33E, M55A, M55L, M55Q, M61V, N125E, G125E, S133T, S157D, Q181E, A205G, P206A, N214D, N228S, N260D, N260E, Q272D, N323E, N336A, N349P, N357E, S407E, S408E, or a combination thereof.

33. The variant polypeptide of claim 32, wherein the amino acid substitution comprises:
i) S407E,
ii) M55L,
iii) A205G and P206A, or
iv) a combination thereof.

34. The variant polypeptide of claim 32, wherein the amino acid substitution comprises:
a) S23E and N125E;
b) Q33E and S157D;
c) Q181E and N260D;
d) N214D and S407E;
e) S23E, Q181E, N260D, Q272E, N323E, N349P, N357E, S407E, and S408D;
f) S23E, Q181E, N228S, N260D, Q272E, N323E, N349P, N357E, and S408D;
g) S23E, Q33E, Q181E, N260E, Q272D, N323E, N349P, N357E, and S407E;
h) S23E, N260E, Q272E, and S407E;
i) S23E, N214D, N260E, Q272E, and S407E;
j) S23E, N214D, N260D, Q272E, and S407E;
k) S23E, N260E, Q272E, N349P, and S407E;
l) S23E, N260E, Q272E, and S407E;
m) M55A and M61V;
n) M55L and M61V;
o) M55Q and M61V;
p) A205G, P206A, and S407E;
q) K9E, V12L, S23M, R31A, E37D, Y39A, D40S, M1021, N125G, K259N, S266D, Q269R, N270Y, E335D, V355A, D361S, G372A, A388A, Y404W, Y406D, S407E, and Y418H; or
r) K9E, V12L, S23M, R31A, E37D, Y39A, D40S, M1021, N125G, A205G, P205A, K259N, S266D, Q269R, N270Y, E335D, V355A, D361S, G372A, A388A, Y404W, Y406D, and Y418H.

35. The variant polypeptide of claim 29, wherein the amino acid modification is amino acid residue 23, 33, 125, 133, 157, 181, 214, 228, 260, 272, 323, 334, 349, 357, 407, 408, or a combination thereof of SEQ ID NO:1.

36. The variant polypeptide of claim 29, wherein the amino acid modification is at amino acid 23, 33, 125, 133, 157, 181, 214, 228, 260, 272, 323, 336, 349, 357, 407, 408 or a combination thereof of SEQ ID NO:1.

37. The variant polypeptide of claim 29, wherein the amino acid modification is at an amino acid residue 55, 61, or a combination thereof of SEQ ID NO:1.

38. The variant polypeptide of claim 29, wherein the amino acid modification is at amino acid residues 205 and 206 of SEQ ID NO:1.

39. The variant polypeptide of claim 29, wherein the variant polypeptide has:
i) alpha-amylase activity,
ii) increased solubility at pH 6.0 compared to the polypeptide of SEQ ID NO:1,
iii) increased solubility at pH 10.0 compared to the polypeptide of SEQ ID NO:1,
iv) increased resistance to protein aging compared to the polypeptide of SEQ ID NO:1,
v) increased specific activity compared to the polypeptide of SEQ ID NO:1, or
vi) a combination thereof.

40. The variant polypeptide of claim 29, wherein the variant polypeptide is a fragment of SEQ ID NO:1.

41. A hybrid polypeptide comprising the variant polypeptide of claim 29 and a second polypeptide having amylase activity.

42. A composition comprising the variant polypeptide of claim 29 and a second enzyme.

43. The composition of claim 42, wherein the second enzyme is selected from the group consisting of: an beta-amylase, a lipase, a second alpha-amylase, a G4-amylase, a xylanase, a protease, a cellulase, a glucoamylase, an oxidoreductase, a phospholipase, and a cyclodextrin glucanotransferase.

44. A method of making a variant polypeptide comprising providing a template nucleic acid sequence encoding the variant polypeptide of claim 29, transforming the template nucleic acid sequence into an expression host, cultivating the expression host to produce the variant polypeptide, and purifying the variant polypeptide.

45. The method of claim 44, wherein the expression host is selected from the group consisting of: a bacterial expression system, a yeast expression system, a fungal expression system, and a synthetic expression system.

46. The method of claim 45, wherein:
i) the bacterial expression system comprises an E coli, a Bacillus, a Pseudomonas, or a Streptomyces;
ii) the yeast expression system comprises a Candida, a Komagataella, or a Saccharomyces, a Schizosaccharomyces; or
iii) the fungal expression system comprises a Penicillium, an Aspergillus, a Fusarium, a Myceliopthora, a Rhizomucor, a Rhizopus, a Thermomyces, or a Trichoderma.

47. Use of the variant polypeptide of claim 29 for:
i) starch processing,
ii) cleaning or washing textiles, hard surfaces, or dishes,
iii) making ethanol,
iv) treating an oil well,
v) processing pulp or paper,
vi) syrup production,
vii) preparing a dough or a baked product prepared from the dough,
viii) in a detergent or personal care product, or
ix) a combination thereof.

48. An animal feed, a detergent, a personal care product, or a baked product comprising the variant polypeptide of claim 29.
